(19) United States
(12) Patent Application Publication
(10) Pub. No.: US 2025/0387589 A1
(43) Pub. Date: Dec. 25, 2025

(54) DYNAMICALLY NEURO-HARMONIZED AUDIBLE SIGNAL FEEDBACK GENERATION

(71) Applicant: DEEPWELL DTX, Las Vegas, NV (US)

(72) Inventors: Michael S. Wilson, Seattle, WA (US); Mark Roemer, Las Vegas, NV (US); Michael Lee Jackson, Buffalo, NY (US); Shahid Ahmad, Las Vegas, NV (US); Thomas R. Glunt, Jr., Los Angeles, CA (US); Ryan J. Douglas, Seattle, WA (US)

(73) Assignee: DEEPWELL DTX, Las Vegas, NV (US)

(21) Appl. No.: 18/879,174

(22) PCT Filed: Jun. 29, 2023
(86) PCT No.: PCT/US2023/069442

§ 371 Date: Dec. 26, 2024

Related U.S. Application Data
(60) Provisional application No. 63/367,235, filed on Jun. 29, 2022.
(60) Provisional application No. 63/485,626, filed on Feb. 17, 2023.

(51) Int. Cl. A61M 21/02; A61M 21/00; G10L 25/63; G16H 20/70

(52) U.S. Cl. CPC A61M 21/02; G10L 25/63; G16H 20/70; A61M 2021/0027; A61M 2021/0088; A61M 2205/3375

(57) ABSTRACT

Apparatus and associated methods relate to a neuro-harmonizing system (NHS). In an illustrative example, the NHS may automatically generate a neuro-harmonizing audible feedback package to induce a non-therapeutic state. The NHS, for example, may receive an input signal from a user device. For example, the NHS may apply the input signal to a state prediction model to identify a current state of the user device. A set of target criterion, for example, may be generated based on the current state. For example, the set of target criterion including transformation parameters configured to transform the input signal into a new signal for inducing a dynamically generated target state based on a user profile. The NHS may, for example, generate an audible feedback package as a function of the input signal and the set of target criterion. Various embodiments may advantageously induce the dynamically generated target state.

### CROSS-REFERENCE TO RELATED APPLICATIONS

This application claims the benefit of U.S. Provisional Application Ser. No. 63/367,235, titled “DEEPWELL MUSIC EXPERIENCE,” filed by Michael S. Wilson, et al., on Jun. 29, 2022, and U.S. Provisional Application Ser. No. 63/485,626, titled “Computer-Implemented Engagement and Therapeutic Mechanisms,” filed by Michael S. Wilson, et al., on Feb. 17, 2023.

This application incorporates the entire contents of the foregoing application(s) herein by reference.

The subject matter of this application may have common inventorship with and/or may be related to the subject matter of the following:
PCT application serial no. PCT/US21/71585, titled “Immersive Medicine Translational Engine for Development and Repurposing of Non-Verified and Validated Code,” filed by Ryan J. Douglas, et al., on Sep. 24, 2021;
PCT application serial no. PCT/US2023/063720, titled “Treatment Content Delivery and Progress Tracking System,” filed by Ryan J. Douglas, et al., on Mar. 3, 2023;
U.S. Pat. No. 11,295,261, titled “FDA COMPLIANT QUALITY SYSTEM TO RISK-MITIGATE, DEVELOP, AND MAINTAIN SOFTWARE-BASED MEDICAL SYSTEMS,” filed by Ryan J. Douglas, on May 28, 2019;
U.S. Pat. No. 11,531,949, titled “FDA COMPLIANT QUALITY SYSTEM TO RISK-MITIGATE, DEVELOP, AND MAINTAIN SOFTWARE-BASED MEDICAL SYSTEMS,” filed by Ryan J. Douglas, on Mar. 1, 2022; and,
U.S. patent application Ser. No. 18/055,754, titled “FDA COMPLIANT QUALITY SYSTEM TO RISK-MITIGATE, DEVELOP, AND MAINTAIN SOFTWARE-BASED MEDICAL SYSTEMS,” filed by Ryan J. Douglas, on Nov. 15, 2022.

This application incorporates the entire contents of the foregoing application(s) herein by reference.

### TECHNICAL FIELD

Various embodiments relate generally to audible control signal generation associated with virtual reality sensory experience.

### BACKGROUND

Music has been an integral part of human culture and society for centuries. In some cases, music may serve various purposes beyond mere entertainment. For example, different types of music may influence human emotions, cognition, and/or overall well-being. Music, for example, may evoke emotional responses (e.g., create a sense of connection, enhance communication between people). Music may sometimes be used as a therapeutic tool in different cultures and throughout history, demonstrating its potential to promote healing, reduce stress, and improve overall quality of life.

In some examples, music may also include therapeutic potential of music to address physical, psychological, and emotional conditions. Music therapy may, for example, include a skilled use of music to facilitate positive changes in an individual. For example, therapeutic applications of music may demonstrate promising results in pain management, stress reduction, mood enhancement, and/or cognitive stimulation.

Calming effects of music may be used in therapeutic applications to address conditions such as anxiety, insomnia, and stress-related disorders. Music may, for example, be carefully selected to facilitate relaxation, emotional regulation, and/or overall well-being. Sometimes, music may be selected by trained professionals having expertise in tailoring music experiences to meet specific needs of individuals. Through the use of calming music, soothing melodies, and rhythm, for example, the selected music may create a serene and supportive environment conducive to healing and/or emotional release.

### SUMMARY

Apparatus and associated methods relate to a neuro-harmonizing system (NHS). In an illustrative example, the NHS may automatically generate a neuro-harmonizing audible feedback package to induce a non-therapeutic state. The NHS, for example, may receive an input signal from a user device. For example, the NHS may apply the input signal to a state prediction model to identify a current state of the user device. A set of target criterion, for example, may be generated based on the current state. For example, the set of target criterion including transformation parameters configured to transform the input signal into a new signal for inducing a dynamically generated target state based on a user profile. The NHS may, for example, generate an audible feedback package as a function of the input signal and the set of target criterion. Various embodiments may advantageously induce the dynamically generated target state.

Various embodiments may achieve one or more advantages. For example, some embodiments may advantageously decouple a user from “regulated breathing” exercises to overcome a natural aversion to self-help techniques and/or a social stigma attached to “meditative” exercises. Some embodiments, for example, may generate vocal tracks of a variety of tempo to advantageously maintain an interest of the user to use the NHS. For example, some embodiments may advantageously allow fast pitch matching and/or beat matching to generate a harmonizing interventional sound. For example, some embodiments may include mini games to advantageously train a response of the user to properly respond in a panic state. Some embodiments, for example, advantageously encourage community involvement within special communities.

The details of various embodiments are set forth in the accompanying drawings and the description below. Other features and advantages will be apparent from the description and drawings, and from the claims.

### BRIEF DESCRIPTION OF THE DRAWINGS

FIG. 1A and FIG. 1B depict an exemplary neuro-harmonizing system (NHS) employed in illustrative use-case scenarios.

FIG. 2A and FIG. 2B are block diagrams depicting an exemplary neuro-harmonizing device (NHD) and an exemplary output generated by the NHD.

FIG. 3 is a flowchart illustrating an exemplary NHA configuration method.

FIG. 4 is a flowchart illustrating an exemplary NHA runtime method for dynamically generating a neuro-harmonized audible feedback signal.

FIG. 5 is a flowchart illustrating an exemplary NHA runtime method for dynamically generating a breathing control signal.

FIG. 6A and FIG. 6B are block diagrams depicting an exemplary multi-dimensional immersive neuro-harmonizing system (MDINHS).

FIG. 7 is a block diagram of an exemplary game-induced neuro-harmonizing system (GINHS).

Like reference symbols in the various drawings indicate like elements.

### DETAILED DESCRIPTION OF ILLUSTRATIVE EMBODIMENTS

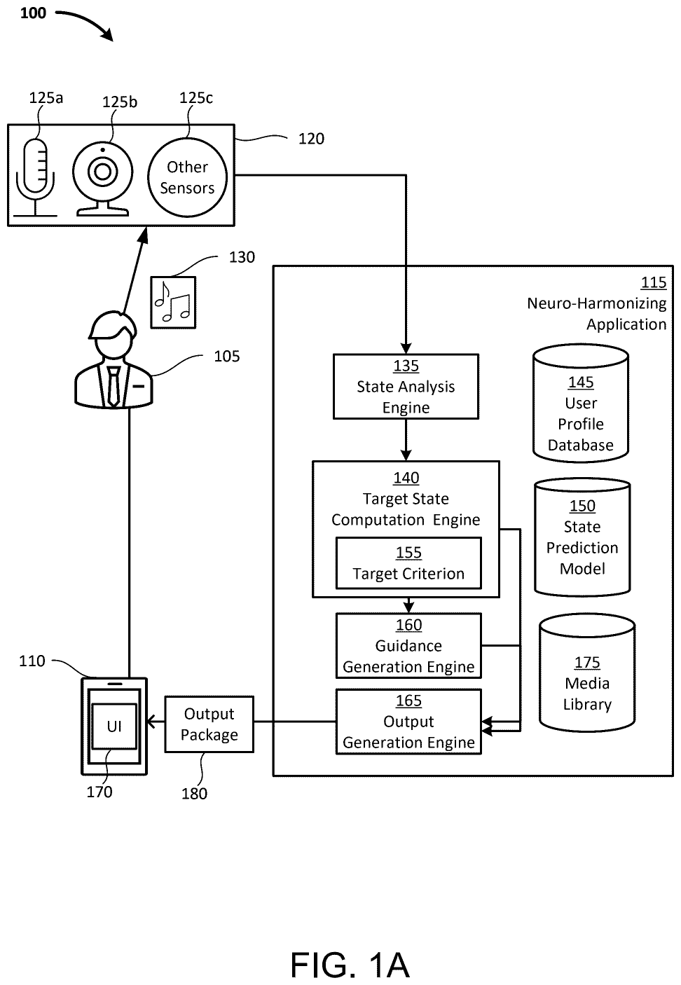

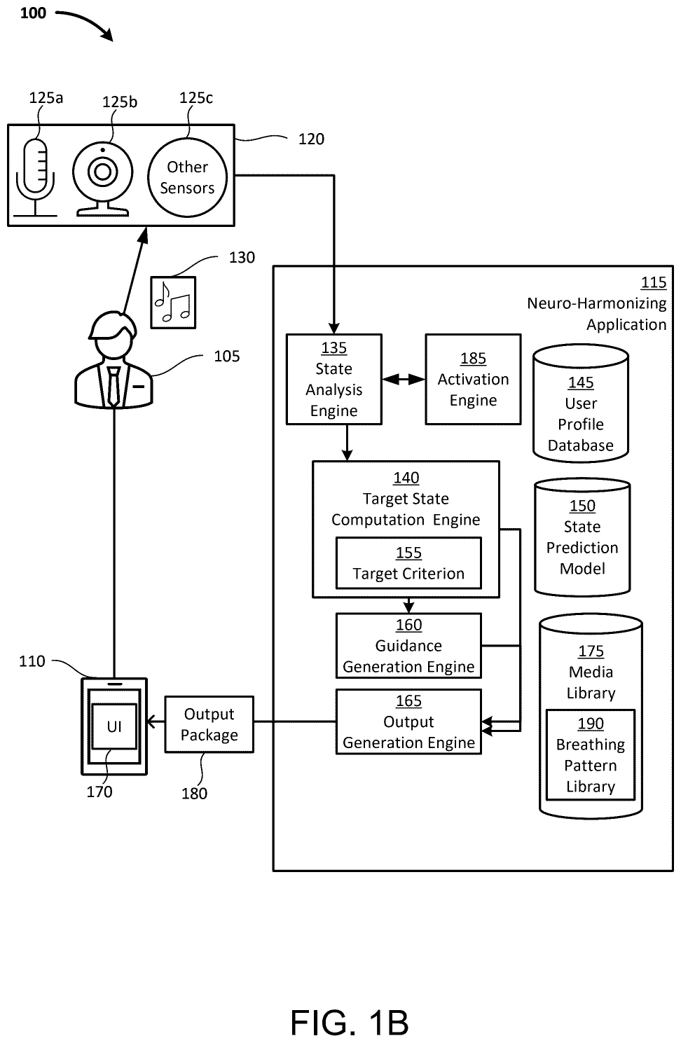

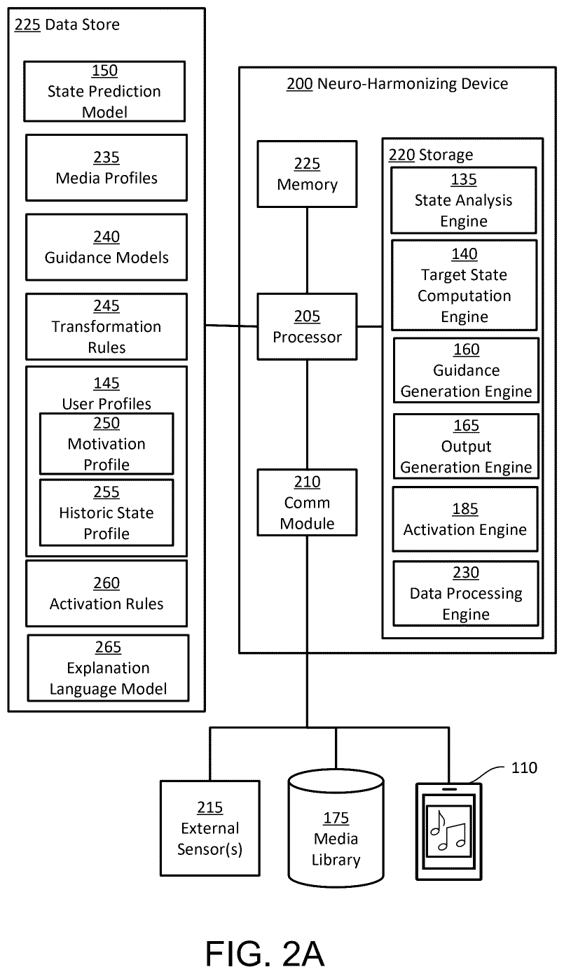

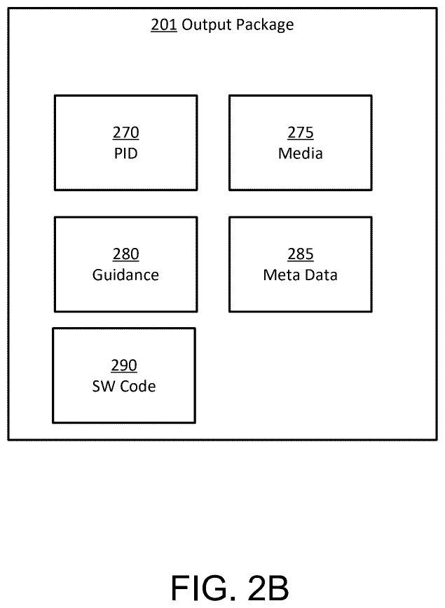

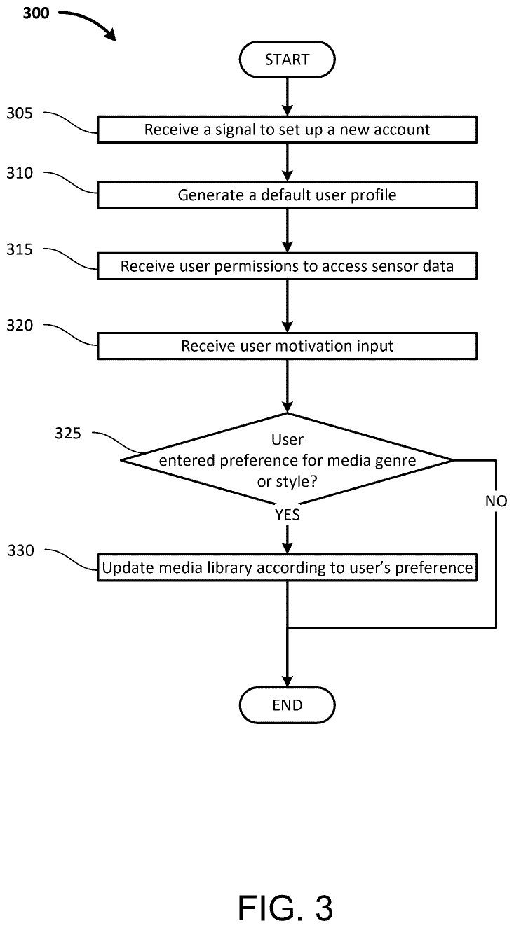

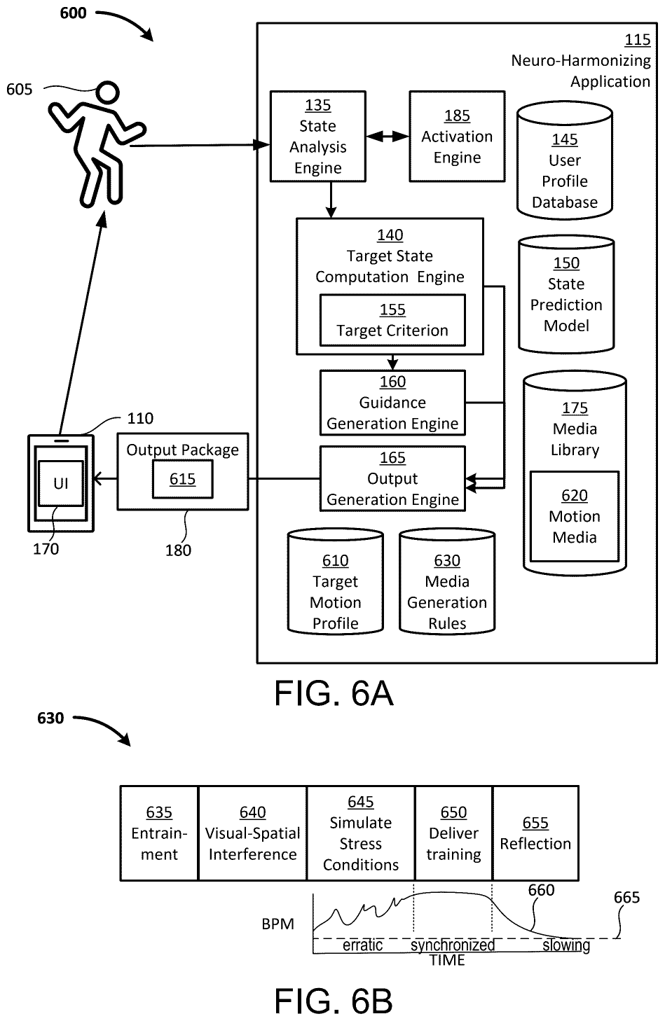

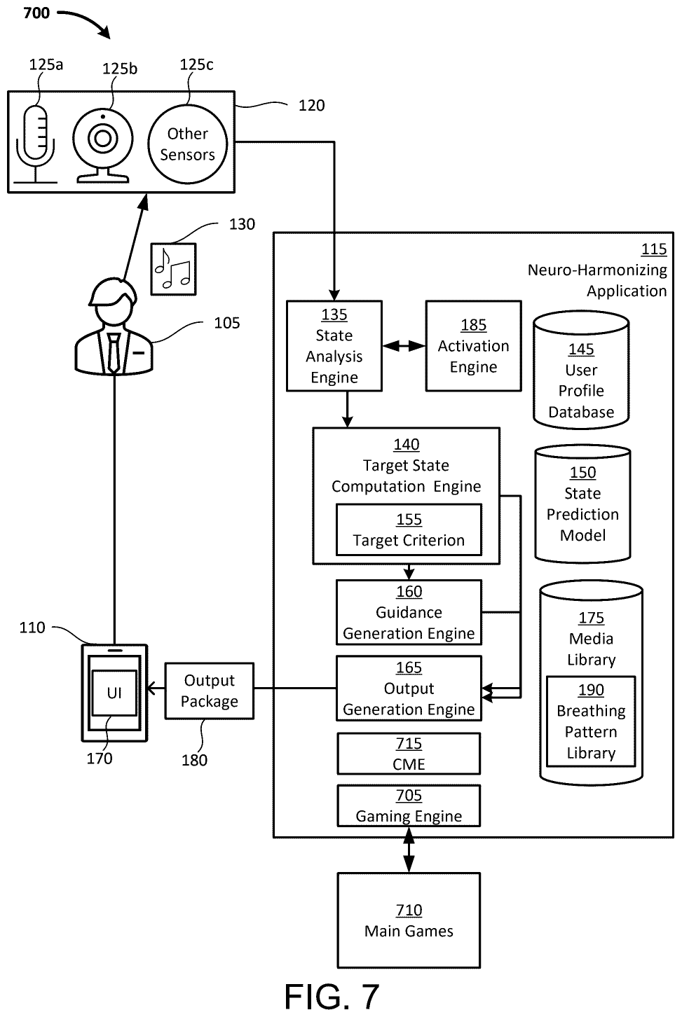

To aid understanding, this document is organized as follows. First, to help introduce discussion of various embodiments, a neuro-harmonizing system (NHS) is introduced with reference to FIGS. 1A-1B. Second, that introduction leads into a description with reference to FIGS. 2A-2B of some exemplary embodiments of a neuro-harmonizing device. Third, with reference to FIGS. 3-5, methods for configuration and running the NHS are described. Fourth, with reference to FIGS. 6A-B, the discussion turns to exemplary embodiments that illustrate an exemplary multimedia-based neuro-harmonizing system. Fifth, and with reference to FIG. 7, this document describes exemplary apparatus and methods useful for integrating the NHS into video games. Finally, the document discusses further embodiments, exemplary applications and aspects relating to the NHS.

FIG. 1A and FIG. 1B depict an exemplary neuro-harmonizing system (NHS) employed in illustrative use-case scenarios. As shown in FIG. 1A, an NHS 100 includes a user 105 and a mobile device 110. In this example, the mobile device 110 is running a neuro-harmonizing application (NHA 115). For example, the mobile device 110 may, for example, include a computer, a game console, a phone, a virtual reality headset, a television, a handheld device, a motion sensor, a camera, and/or a car screen console.

The NHA 115, for example, may include a music playback application. For example, the NHA 115 may include a game application. For example, the NHA 115 may include an interactive application. In some implementations, the NHA 115 may include a non-therapeutic application. For example, the NHA 115 may include non-therapeutic interactive mechanisms (NTIM) (e.g., toning and/or guided sound making mechanisms). The NTIM may, for example, advantageously decouple the user 105 from “regulated breathing” exercises. The NTIM may, for example, advantageously help overcome a natural aversion to self-help techniques and/or a social stigma attached to “meditative” exercises. Various embodiments may advantageously provide a solution to a problem of resistance to use self-help or “good for your activities” that may be perceived as unfavorable, not fun, and/or a chore.

In this example, the NHS 100 includes a sensor module 120. As shown, the sensor module 120 may include an audio sensor 125a, a camera 125b, and other sensors 125c. The NHA 115 receives sensor data from the sensor module 120. For example, the sensor module 120 may include sensor(s) that may be operably (wirelessly) coupled to the mobile device 110. For example, the mobile device 110 may include some or all of the sensor(s) of the sensor module 120. In this example, the sensor module 120 may receive a voice clip 130. For example, the user 105 may sing to produce a voice clip 130 to be captured by the audio sensor 125a. In various implementations, after receiving the voice clip 130, the NHA 115 may generate a neuro-harmonizing tone to be played back at the mobile device 110 based on the voice clip 130. For example, the audio sensor 125a may include, for example, dynamic microphones, carbon microphones, ribbon microphones, and/or piezoelectric microphones.

In various implementations, the NHA 115 may receive exhalation sounds(ES) from the other sensors 125c and/or the audio sensor 125a. The ES may, for example, be used to establish breathing patterns and determine rates of breath as an input for the NHS 100. Various embodiments may solve a problem of easily detecting breath without the need for additional hardware, greatly increasing access to care to anyone with a device that can detect noise or sounds.

In this example, the NHA 115 includes a state analysis engine (SAE 135) and a target state computation engine (TSCE 140). The SAE 135 may, for example, analyze the voice clip 130 by comparing a vocalization of the user 105 and a target model based on a user profile. In this example, the NHA 115 includes a user profile database 145 and a state prediction model 150. For example, the user profile database 145 may include demographic information of the user 105. For example, the user profile database 145 may include historical interaction results of the user 105. The user profile may, for example, include a categorization, a rating, an indicator, memories, timelines, a data set of the user's preferences, and/or an analysis of the user. The user profile may, for example, include categorization, memories, timelines, a rating, a data set of the group's preferences, and/or an indicator of a group of musical users signing together. The user profile may, for example, include a categorization, a rating, memories, timelines, a data set of the clan's preferences, an indicator, and/or a clan of musical users (e.g., users of the NHA 115 across a user-defined, geographical, and/or demographic group).

In some implementations, the SAE 135 may selectively apply the state prediction model 150 based on a user profile of the user 105 from the user profile database 145. For example, the SAE 135 may generate a current state of the user 105 based on the voice clip 130. For example, the current state may include a biological (e.g., physical health, emotional health) state of the user 105. For example, the current state may include an analytic state of the voice clip 130. For example, the state prediction model 150 may include a tonal analysis including analysis of a pitch, a volume, a vibrato, and/or other music elements of the voice clip 130.

After generating the current state of the user 105, for example, the TSCE 140 may generate a target state for the user 105. As shown, the TSCE 140 generates a target criterion 155 based on the current state. For example, the target criterion 155 may include a set of target criterion. For example, the set of target criterion may include transformation parameters of a sound clip. Based on the transformation parameters, the voice clip 130 may be altered in pitch (e.g., a frequency transformation), intensity (e.g., an amplitude transformation), in beats (e.g., a pattern transformation), and/or environment (e.g., a background generative transformation). In some implementations, the target criterion 155 may include a set of target key notes and or rhythms identified in the voice clip 130.

In some implementations, the TSCE 140 may determine the set of target criterion as a function of a predicted state generated by applying the state prediction model 150 based on the current state and the user profile. For example, the state prediction model 150 may include an assessment of likelihood of the target criterion 155 for achieving a predicted state. For example, the user profile may include a target state of an increased engagement from the user 105. As an illustrative example without limitation, based on the target state, the TSCE 140 may generate the target criterion 155 to transform the voice clip 130 into an exciting voice that may be determined to be likely (e.g., higher than a predetermined threshold) to increase engagement of the user 105.

In this example, the NHA 115 includes a guidance generation engine (GGE 160) and an output generation engine (OGE 165). For example, the OGE 165 may generate transformed sound data to the mobile device 110 as a function of the voice clip 130 and the target criterion 155. The OGE 165, in some implementations, may generate an interventional sound based on the voice clip 130. For example, the OGE 165 may modulate, as a function of the target criterion 155, the pitch, the volume, the vibrato, and/or hard hits for beat of the voice clip 130 to match with a target harmonized tone. For example, the harmonized tone may be determined using the state prediction model 150 and the user profile database 145 to include a probability higher than a predetermined threshold in achieving the target state.

The predetermined threshold may, for example, be at least 60%. The predetermined threshold may, for example, be at least 70%. The predetermined threshold may, for example, be at least 80%. The predetermined threshold may, for example, be at least 95%.

In various embodiments, various target states may be generated. The target state may, for example, include an increase in the muscles of the diaphragm. The target state may, for example, improve the amount of cerebrospinal fluid spinal fluid transferred to the brain and may provide greater engagement and may aid in neural transmission of neural chemicals (e.g., which may enhance the creation, preferential use, and/or efficiency of certain neural pathways). The target state may include, for example, to encourage a user to breathe a certain rhythm. For example, the target state may be motivational. Being included in a singing or vocalization and therefore, for example, part of a harmonious and/or connected action can, by way of example and not limitation, entice the user 105 to continue the action for reasons of perceived socialization, increased health and wellbeing, personal satisfaction, and/or other such engagements.

As an illustrative example, the OGE 165 may generate a harmonizing tone based on the voice clip 130 and the target criterion 155. For example, the OGE 165 may generate a pitch-corrected sample of the voice clip 130. The pitch-corrected sample may be subtly fed back to the user 105. This feedback may, for example, be configured to induce the target state (e.g., to make the user 105 feel like they are singing beautifully without requiring the user to actually be on pitch). In various embodiments, the OGE 165 may generate the interventional sound to, for example, help a user in ‘harmonizing with your best self’ and/or being ‘led by your best self.’ For example, the user 105 may be induced with confidence and/or a sense of calmness when his/her voice sounded good and in control.

The GGE 160, for example, may generate a guidance to the user 105 based on the target criterion 155. In some implementations, the guidance may include a guidance to perform a voluntary action. For example, the voluntary action may include singing along with a song. For example, the voluntary action may include reciting a poem. For example, the guidance may include an action instruction (e.g., “sing this song like you are in a national park”), an index of keys, auto-corrective key instructions, and/or rhythm correction prompts. For example, the guidance may include a suggestion of an activity. In some implementations, the GGE 160 may select from a media library 175, a media (e.g., a video clip, a voice clip, a game) to be played on the mobile device 110. For example, the media library 175 may include an internal music database, an external music database, a streaming platform, an index of songs and/or a cloud-based song catalog.

In some implementations, the media library 175 may, for example, include media created by a few (e.g., predominant, registered, or otherwise qualified) artists. For example, the NHS 100 may include an artist qualification criterion based on a predetermined (e.g., initial) selection of artists. The selection may, for example, be configured to include multiple (e.g., several) genres appealing to multiple age groups. In some implementations, the media library 175 may, for example, include playlists offered (e.g., verified) to users of the NHA 115. The playlists may, by way of example and not limitation, be curated by an administration module of the NHS 100 (e.g., automatically). For example, the playlist may be created by (invited) artists, and/or by a user community. User-created playlists may, for example, advantageously provide a strong possibility of social engagement with sharing and discovery of user-created playlists.

As shown, the mobile device 110 includes a user interface 170. The user interface 170 may display the guidance generated by the GGE 160. In this example, an output package 180 is generated by the OGE 165. For example, the OGE 165 may combine a guidance package and the target criterion 155 to generate the output package 180. For example, the user interface 170 may display instructions and/or guidance in the output package 180. In some implementations, the mobile device 110 may, upon receiving the output package 180, generate an audible feedback signal to the user 105 based on the voice clip 130. In some implementations, the user interface 170 may display a visual display (e.g., visual guidance, a visual pattern related to the target state) based on a received output package.

In some implementations, the user interface 170 may also include user input (e.g., a chatbot) for the user 105 to provide direct feedback to the NHA 115. As an illustrative example without limitation, the GGE 160 may, based on the target criterion 155 selected a song from a media library 175 to be sung by the user 105. For example, the user 105 may use the user interface 170 to deny the request and/or provide a reason (e.g., “I do not like this song under rainy weather.”). For example, the NHA 115 may receive the user feedback and store the feedback to the user profile database 145.

In various embodiments, the GGE 160 may generate breath cues to promote a target breathing pattern of the user 105. For example, the output package 180 may include the breath cues in a text format. For example, the output package 180 may include the breath cues in a multi-media (e.g., in audio, in video, in audio combined with video, in audio combined with video and text). For example, the breath cues may be generated to be synchronized in time with an interventional sound clip generated by the OGE 165 to the mobile device 110. As an illustrative example, the GGE 160 may generate a breath cue to include a visual indicia that inspires breath. In another example, the GGE 160 may generate a breath cue to include an audible feedback signal that inspires breath.

Various implementations may, for example, adjust (e.g., dynamically, based on feedback, according to predetermined profiles) how frequently this prompt happens so that it doesn't overwhelm the experience or take away from the music. Various implementations may, for example, utilize a waveform generator that analyzes a soundtrack and creates a visual representation of the music and identifies the breath pattern to follow.

In some implementations, an output visual may be shared. For example, a player may play a song with the player's breath and the song breathes the player in and up through some interactions (e.g., in a gaming environment). As a result, for example, the player may create an output that may be shared with friends. In a social aspect, after completing a song, an artifact/playback of the player's experience can be shared (e.g., upon player request/permission) with friends (e.g., inside an associated game).

In some implementations, the NHA 115 may be activated by the user 105 via the user interface 170. In some implementations, the NHA 115 may be a background process listening to the sensor module 120 (e.g., with user permissions). For example, the NHA 115 may generate the output package 180 to generate an interventional sound to the mobile device 110 based on sensor data received from the sensor module 120. In some implementations, the NHA 115 may be embedded and/or associated with other applications of the mobile device 110. For example, when a current state is determined to match a predetermined state, the NHA 115 may intervene and generate the output package 180 to the user 105.

As shown in FIG. 1B, the NHA 115 includes an activation engine 185. For example, the activation engine 185 may receive the current state from the SAE 135. In response to a predetermined activation criterion being satisfied, the activation engine 185 may generate an activation signal to the SAE 135 to begin a process to generate the output package 180. For example, the activation engine 185 may determine that the user 105 is anxious based on, for example, a breathing pattern received from the audio sensor 125a and/or the camera 125b. For example, breathing patterns may be measured from the other sensors 125c based on haptic, EEG, ECG, and/or pupil response.

In some implementations, the user 105 may, for example, not be prompted at all after starting a program while having the NHA 115 observing the breathing pattern of the user 105. For example, the NHA 115 may use the sensor module 120 to analyze the user. In a normal mode, the NHA 115 may, for example, log behaviors and/or improvements of the user 105 in the user profile database 145.

When the activation criteria are met, for example, the NHA 115 may enter an intervention mode. For example, the activation engine 185 may interrupt a currently executing routine to enter the intervention mode. Various embodiments of the activation engine 185 are described with reference to PCT application serial no. PCT/US2023/063720, which shares at least one inventor with this application, titled “Treatment Content Delivery and Progress Tracking System,” filed on Mar. 3, 2023, specifically, in FIGS. 1A-B, 4-6, and 10, and [0040-44], [0114-15], and [0144-50]. This application incorporates the entire contents of the foregoing application(s) herein by reference.

In this example, the media library 175 includes a breathing pattern library 190. For example, the breathing pattern library 190 may include breathing patterns designed to induce a target state of breathing for the user 105. For example, in a gaming environment, a game player may, for example, be encouraged by the output package 180 to create particular breathing patterns through vocalization, toning, and/or non-toning biofeedback and/or pacing. For example, based on the target criterion 155, the OGE 165 may retrieve a breathing pattern from the breathing pattern library 190. For example, the breathing pattern may include an exhalation frequency selected to have a calming effect for the user 105 based on the user profile. For example, the GGE 160 may generate an instruction (e.g., in text, audio, video, or a combination thereof) to be displayed at the user interface 170.

Various breathing patterns may be used to achieve the target states. The user may, for example, be prompted to sing a certain musical note and/or prompted song. The user may, for example, be prompted to hum a certain musical note and/or prompted song. The user may, for example, be encouraged to utter a chant or other such words which may be a poem, spell, or charm. As an illustrative example, the NHA 115 may prompt the user 105 to hum a quick tempo phrase may be used while having a calming effect. In some embodiments, the NHA 115 may track a breathing cadence, gestures, humming pitch, humming Intensity, and humming accuracy. In some implementations, the NHA 115 may receive data from the camera 125b to detect whether a smooth movement of hand is detected to indicate pitch or vibrato change. For example, fast movement of the hand may indicate rhythmic reinforcement.

In various embodiments, a method to generate a target audible output data structure (e.g., the output package 180) may include receiving a signal (e.g., the voice clip 130, an activation signal, sensor data from the sensor module 120) from a user device (e.g., the mobile device 110). For example, the signal may include a voice clip generated by the user 105. The method may include identifying a current state of the user by applying a state prediction model (e.g., the state prediction model 150) to the voice clip. For example, the method may include identifying a set of target criterion (e.g., the target criterion 155) based on the current state. For example, the set of target criterion may include transformation parameters of a sound clip. For example, the transformation parameters may include frequency transformation, amplitude transformation, background generative transformation, and pattern transformation. For example, the set of target criterion may be determined by a state control model (e.g., the TSCE 140) configured to generate a predicted state based on the current state and the set of target criterion. The method may, for example, include generating an interventional sound as a function of the voice clip and the set of target criterion. For example, the interventional sound may have a probability above a predetermined threshold to induce the predicted state.

For example, the method further includes transmitting an instruction (e.g., to be included in the output package 180) to the user device. For example, the instruction may include a guidance for performing a voluntary action (e.g., perform a vocal action).

In some examples, the intervention sound may be generated by requesting an input sound clip from the user based on a generated guidance. For example, the GGE 160 may generate an instruction to request an input sound clip (e.g., for reciting a poem provided). For example, after the requested sound clip is received, the OGE 165 may apply the set of target criterion. The OGE 165 may, for example, generate an audible feedback signal to the mobile device 110.

For example, the background generative transformation may include transforming a background noise of a sound clip to include sound effects. For example, the OGE 165 may generate a background noise as if the user 105 is singing and/or vocalizing as part of a choir and/or group of other users. The users may, for example, hear their voice as a modulate and/or as a changed sound, a different pitch, and/or interrupted as one of any number of instruments and/or animal sounds. A user may hear the output of the device and may, for example, be encouraged to continue to use the device because the user enjoys the sound of their voice corrected to be on key or otherwise modified to match the narrative of the game play or activity. In some examples, the sound effects include background effects that sound like a video game. In some examples, the sound effects include background effects that sound like a music band.

In some implementations, the predicted state may include a target breathing pattern. For example, a request (e.g., generated by the GGE 160) of an audio input from the user may include an instruction to perform a voluntary action related to a controlled vocal track. For example, the controller vocal track may be stored in the breathing pattern library 190. For example, the voluntary action may include humming along with the controlled vocal track such that the breathing pattern may achieve the predicted state.

FIG. 2A and FIG. 2B are block diagrams depicting an exemplary neuro-harmonizing device (NHD) and an exemplary output generated by the NHD. As shown in FIG. 2A, an NHD 200 includes a processor 205. The processor 205 may, for example, include one or more processors. The processor 205 is operably coupled to a communication module 210. The communication module 210 may, for example, include wired communication. The communication module 210 may, for example, include wireless communication. In the depicted example, the communication module 210 is operably coupled to the mobile device 110 and the media library 175. As shown, the communication module 210 is also operably coupled to external sensor(s) 215. For example, the external sensor(s) 215 may include some or all of the sensors in the sensor module 120. For example, the sensor(s) 215 may be integrated in the mobile device 110. For example, the sensor(s) 215 may be pluggable to the mobile device 110. For example, the sensor(s) 215 may be connected (wirelessly) to the mobile device 110. For example, the NHD 200 may receive sensor data associated with a user (e.g., the user 105) from the sensor(s) 215.

The processor 205 is operably coupled to a memory module 220. The memory module 220 may, for example, include one or more memory modules (e.g., random-access memory (RAM)). The processor 205 includes a storage module 225. The storage module 225 may, for example, include one or more storage modules (e.g., non-volatile memory). In the depicted example, the storage module 225 includes the SAE 135, the TSCE 140, the GGE 160, the OGE 165, and the activation engine 185. As described with reference to FIGS. 1A-B, the SAE 135, the TSCE 140, the GGE 160, the OGE 165, and the activation engine 185 may generate the output package 180 to induce a target state in the user 105. Various embodiments of the output package 180 are discussed with reference to FIG. 2B.

In this example, the memory module 220 also includes a data processing engine (DPE 230). For example, the DPE 230 may process sensor data received from the sensor(s) 215. For example, the DPE 230 may preprocess the received sensor data to improve quality of the SAE 135. For example, the DPE 230 may remove noise from the received sensor data. For example, the DPE 230 may generate a frequency domain vector of the sensor data. For example, the DPE 230 may perform a Fast Fourier Transform (FFT) on the received data before passing the data to the SAE 135. In some implementations, the DPE 230 may dynamically (e.g., continuously) generate an input vector to the SAE 135 until a predetermined output quality threshold is reached.

For example, when a user's voice is recorded for 3 seconds, an output of the SAE 135 may have a low quality because of the lack of data. For example, the DPE 230 may determine that the data is insufficient. In some examples, the DPE 230 may combine the previous input and additional 7 seconds (total 10 second) of data to be sent to the SAE 135 for determining a current state of the user. For example, if the quality (e.g., a f-score) of the SAE 135 this time is higher than a predetermined threshold, the NHA 115 may be allowed to proceed to a next step to generate the output package 180.

In the depicted example, the storage module 225 includes the TSCE 140 and the state prediction model 150. The storage module 225 also includes media profiles 235. For example, the media profiles 235 may include metadata and/or characteristics of media stored in the media library 175. For example, the media profiles 235 may genre of the media. For example, the media profiles 235 may include a tempo of the media. For example, the media profiles 235 may include an emotional meaning (e.g., positive, negative, neutral) of the media. In some implementations, the NHS 100 may include an automatic audio analysis engine (e.g., using a spectrometer, beats per minute, waveform) to automatically categorize tracks in the media library 175, by way of example and not limitation, by genre, tone, and/or mood.

For example, the OGE 165 may use the media profiles 235 to select a media from the media library 175 based on the target criterion 155.

The storage module 225 includes guidance models 240 and transformation rules 245. For example, after the media is selected, the GGE 160 may generate an instruction to the user 105 using the guidance models 240. For example, the guidance models 240 may be a trained artificial intelligence model to generate guidance in visual, audio, text, or a combination thereof.

For example, the TSCE 140 may generate the target criterion 155 based on a received voice clip 130 and the transformation rules 245. For example, the transformation rules 245 may include rules (e.g., software code) to harmonize an incoming voice clip. In some implementations, the transformation rules 245 may consider a preference indicated in the user profile to determine a target set of transformation rules applicable to the mobile device 110.

As shown, the user profile database 145 includes a motivation profile 250 and a historic state profile 255. For example, the motivation profile 250 may include a qualitative and/or quantitative motivation of a corresponding user. For example, the motivation profile 250 may include a target emotional state (e.g., a calm state). For example, the motivation profile 250 may include a target breathing pattern (e.g., at most 12 breathing cycles per minute). For example, the motivation profile 250 may be time-dependent (e.g., achieve a calm state 80% of the time after 8 pm). In some implementations, the TSCE 140 may generate the target criterion 155 based on the motivation profile 250. In some implementations, the activation engine 185 may use the motivation profile 250 to generate an activation signal. For example, the NHD 200 may update activation rules 260 based on the motivation profile 250. For example, the activation engine 185 may generate the activation signal based on the activation rules 260.

The historic state profile 255, for example, may include historical states of the user 105. For example, the NHD 200 may log behaviors of the user 105 to the historic state profile 255. In some examples, the NHD 200 may also log user feedback to the historic state profile 255. For example, the state prediction model 150 may be updated based on the historic state profile 255. For example, the state prediction model 150 may be trained using machine learning techniques using the historic state profile 255.

In some implementations, the NHD 200 may be configured to interactively communicate with a user. For example, the user may inquire about his/her (non-therapeutic) progress in the NHA 115. For example, a non-therapeutic progress may include changes in breathing patterns. For example, the non-therapeutic progress may include % of calmness in a period of time. For example, the user may inquire further explanation on a received guidance. In this example, the storage module 225 includes an explanation language model 265. For example, the explanation language model 265 may include a large language model (LLM). In some implementations, the explanation language model 265 may be trained to answer questions specific to neuro-harmonizing feedback to the user. For example, the explanation language model 265 may further enhance a non-therapeutic result to the user by responsively answering the user's questions based on a corresponding user profile in the user profile database 145.

As shown in FIG. 2B, an exemplary output package 201 (e.g., the output package 180) is shown. For example, the output package 201 may be generated by the NHA 115. In this example, the output package 201 includes a package identification (PID 270), a media content 275, and a guidance content 280. For example, the NHA 115 may identify the output package 201 using the PID 270. For example, the NHA 115 may log reactions and results of the output package 201 using the PID 270. The media content 275, for example, may be audio feedback generated by the OGE 165. In some implementations, the media content 275 may also include a selected media retrieved by the OGE 165. In some examples, the media content 275 may include a vocal clip configured to induce a target breath pattern.

The GGE 160 may generate the guidance content 280, for example. The guidance content 280 may include instructions to perform a voluntary action to induce a predicted state.

The output package 201 also includes a meta data 285 and a software code 290. In some implementations, the meta data 285 may include “rewards” assigned to the user if the output package 201 is performed. As an illustrative example, perfection in a visual-audio experience may include hitting all cues on time (e.g., and on pitch) resulting in an almost identical visual reward for everyone and/or every time a player plays. Some embodiments may include a randomizer in the back end to keep things fluid and interesting from person to person and day to day. When a player does great, for example, various things may be configured to respond to ‘great.’ Sometimes, for example, the response may include big changes in color. A response may, for example, include introduction of a fractal. A response may, for example, include randomness to what ‘cool’ things happen when, such that there is some luck of the draw as to what a player gets. Such embodiments may, for example, advantageously increase ‘anticipation’ of players.

In some implementations, the assigned rewards may, for example, include a model based on a time of day and the calendar. “So, you might still get emit x at x due to this input, but it is a different foundation from a pool.” For example, on the back end, various embodiments may load in new visual experiences like finding new ways to break the images up into fractals, having them spin and swirl.

In some implementations, the meta data 285 may include artist-provided information. For example, the artist-provided information may include an album of music in stem form and some artist-provided art for visual display. For example, the meta data 285 may include beat and pitch information for a media track. In some examples, the meta data 285 may advantageously allow fast pitch matching and/or beat matching to generate a harmonizing interventional sound.

In some implementations, the meta data 285 may include a key point of entry for the output package 201 to be interrupting a game. In some implementations, the software code 290 may be executed by the mobile device 110 as a “mini-application” within the user interface 170. For example, the software code 290 may include a mini-game. For example, the software code 290 may include a survey to request feedback from the user 105.

FIG. 3 is a flowchart illustrating an exemplary NHA configuration method 300. For example, the method 300 may be performed by the NHA 115. For example, when the NHA 115 is initiated for the first time, the method 300 may be used to set up a user account for a new user (e.g., the user 105). In this example, the method 300 begins when a signal to set up a new account is received in step 305. For example, the user 105 may run the NHA 115 for a first time using the mobile device 110. Next, in step 310, a default user profile is generated. For example, a default user profile may be generated for the user 105 to be saved in the user profile database 145. In step 315, user permissions to access sensor data are received. For example, the NHA 115 may request various permissions to access the sensor module 120 for further operations.

After the user permissions are received, a user motivation input is received in step 320. For example, the NHA 115 may generate a user interface (e.g., as part of the user interface 170) to receive the user motivation input. For example, the user interface may include a survey. For example, the user interface may include functions for the user to upload various records (e.g., including but not limited to medical records) to determine the motivation profile 250 of the user

In a decision point 325, it is determined whether the user has entered a preference for media genre or style. For example, the user 105 may use the user interface 170 to select one or more preferred genres. In some implementations, the NHA 115 may generate the preference based on a playlist provided by the user 105. If it is determined that the user has entered a preference for media genre or style, in step 330, the media library 175 is updated according to the user's preference, and the method 300 ends. If, in the decision point 325, it is determined that the user has not entered a preference for media genre or style, the method 300 ends.

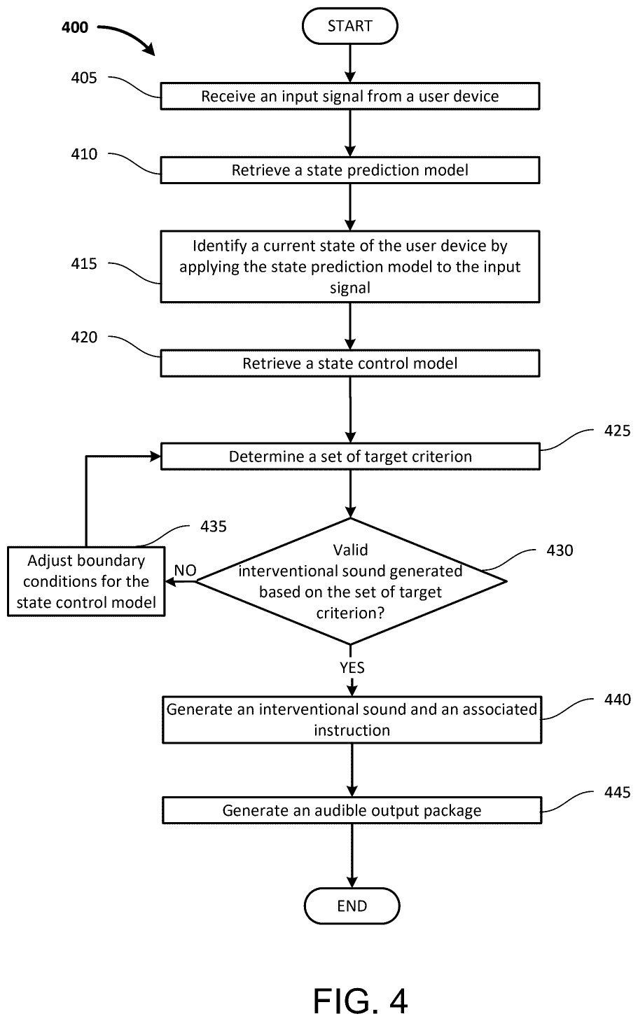

FIG. 4 is a flowchart illustrating an exemplary NHA runtime method for dynamically generating a neuro-harmonized audible feedback signal. For example, the method 400 may be performed by the NHD 200 to automatically generate a neuro-harmonizing audible feedback package to induce a non-therapeutic state. In this example, the method 400 begins in step 405 when an input signal is received from a user device. For example, the signal may include the voice clip 130. In step 410, a state prediction model is retrieved. For example, the state prediction model 150 may be retrieved. For example, the state prediction model 150 may be configured to generate a predicted state as a function of the input signal.

Next, a current state of the user device is identified by applying the state prediction model to the input signal in step 415. For example, the SAE 135 may apply the voice clip 130 to the state prediction model 150. In step 420, a state control model is retrieved. For example, the TSCE 140 may be retrieved. For example, the TSCE 140 may be configured to generate a predicted state as a function of the current state and a control input. For example, the predicted state comprises a target state of the user and a target probability of achieving the target.

In step 425, a set of target criterion is determined. For example, the TSCE 140 may generate the target criterion 155 based on the current state and a predicted state. For example, the target criterion 155 may include transformation parameters of a sound clip. For example, the transformation parameters may include frequency transformation, amplitude transformation, background generative transformation, and pattern transformation of a sound clip. For example, transforming a background noise of a sound may include using a generative model to generate a replacement background noise according to a user profile.

In a decision point 430, it is determined whether a valid interventional sound is generated based on the set of target criterion. For example, the OGE 165 may generate the interventional sound based on the target criterion 155. In some examples, the generated sound may be non-audible (e.g., having too big or too small amplitude, having a frequency out of human hearing and/or singing range). If a valid interventional sound is not generated based on the set of target criterion, in step 435, boundary conditions for the state control model is adjusted, and the step 425 is repeated. For example, the boundary condition may be adjusted from requiring a breathing frequency of 3 per minute to 10 per minute. For example, the boundary condition may be adjusted from requiring a calm emotional state within 10 seconds to achieving the calm emotional state in 5 minutes.

If a valid interventional sound is generated, in step 440, an interventional sound and an associated instruction are generated. For example, the GGE 160 may generate the guidance based on the generated media from the OGE 165. In step 445, an audible output package is generated, and the method 400 ends. For example, the output package 180 may be generated to the mobile device 110.

In some implementations, other output packages may be generated. For example, a video output package may be generated. For example, a gaming package (e.g., to be used in a game) may be generated. For example, a text package may be generated.

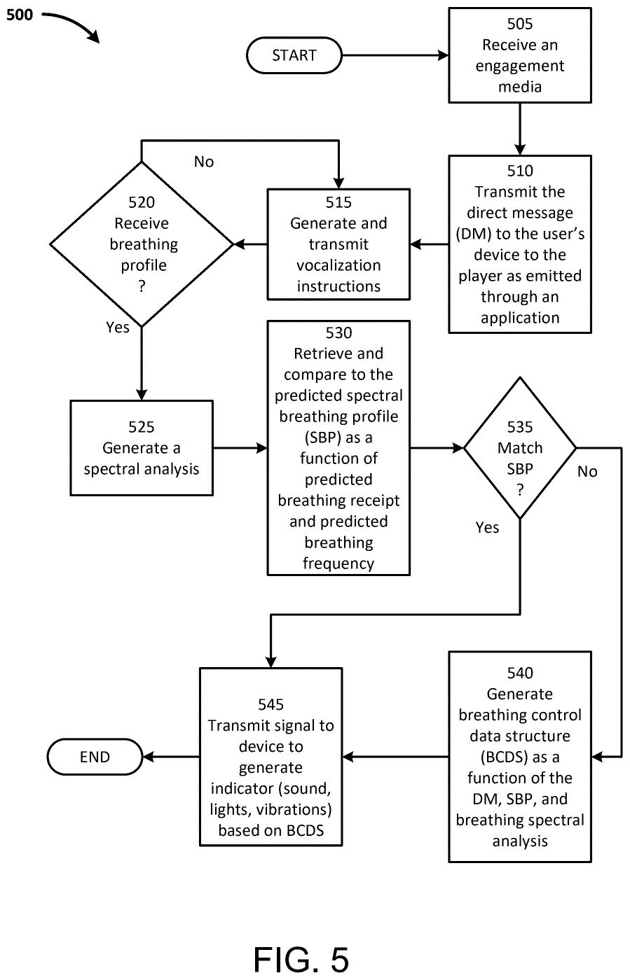

FIG. 5 is a flowchart illustrating an exemplary NHA runtime method 500 for dynamically generating a breathing control signal. In step 505, a dynamic breathing therapeutic mechanism (DBTM) receives an engagement media. For example, the NHA 115 may include the DBTM. For example, the engagement media may include sensor data received from the sensor module 120. For example, the engagement media may include the voice clip 130. The engagement media may, for example, interact with a neurological condition. In step 510, the DBTM transmits a direct message (DM) to a user device through an application launched on the device. For example, the NHA 115 may transmit a message to the mobile device 110 through the user interface 170. In step 515, the DBTM generates and transmits vocalization instructions to the user. For example, the OGE 165 may generate the instruction to the user as the output package 180. The user may, for example, be prompted to sing a certain musical note and/or prompted song. The user may, for example, be prompted to hum a certain musical note and/or prompted song.

In step 520, if the DBTM is able to receive the breathing profile from the vocalization instructions, the DBTM generates a spectral analysis. For example, the spectral analysis may be generated by the SAE 135. If the DHTM is not able to receive a complete breathing profile from the user device, the DHTM prompts another set of vocalization instructions. For example, the OGE 165 may generate a second set of vocalization instructions. The second set of vocalization instructions may, for example, be the same as the first set. The second set of vocalization instructions may, for example, include rhythms not collected in the first vocalization instructions. For example, the prompted song may change depending on the collected data from the first set of instructions to form a complete breathing profile of the user.

In step 525, the DBTM generates a spectral analysis. Next, in step 530, the DHTM retrieves a predicted spectral breathing profile (SBP). For example, the predicted SBP may be generated by the TSCE 140. The DBTM compares the user's SBP with the predicted SBP as a function of the predicted breathing receipt and predicted breathing frequency. The predicted breathing receipt is based on a master key of the breathing frequencies that may, for example, include an engagement value. The predicted breathing frequency is based on a desired breathing pattern. For example, the desired breathing pattern may be generated based on the motivation profile 250 and the current state of the user 105. Breathing training may, for example, increase the muscles of the diaphragm. Breathing training may, for example, improve the amount of cerebrospinal fluid spinal fluid transferred to the brain. Cerebrospinal fluid flow is impaired in numerous neurodegenerative diseases, such as Alzheimer's, stroke, Parkinson's, and multiple sclerosis. Vocalization therapy to encourage a user to breathe a certain rhythm may, for example, affect the amount of cerebrospinal fluid flow to the brain. Breath control therapy may, for example, create other medical effects, such as increasing blood flow, increasing cognitive function, and/or increasing brain hormones such as serotonin, dopamine, endorphins, and/or oxytocin. The comparison of the predicted breathing receipt and the SBP may, for example, allow the DBTM to correct the user's breathing rhythm to align with the predicted breathing rhythm.

In step 535 the DBTM determines whether the user's SBP matches the predicted SBP. If the user's breathing pattern does match the predicted SPBS, proceed to step 540, where the DBTM generates a breathing control data structure (BCDS) as a function of DM, SBP, and breathing spectral analysis.

If the user's breathing frequency matches the SBP and/or after step 540, then the program in step 545 generates an indicator of approval in the form of sound, light, or touch, such as vibration, to the user. For example, the indicator may be displayed at the user interface 170. The indicator may, for example, be a visual cue of “Good Job! and/or “Super!”. A user may view the indicator output of the device and may, for example, be encouraged to continue to use the device because the user enjoys the visual indicator.

In some implementations, a breathing control application may, for example, include a virtual sensory safe space designed to reduce over stimulation in the game. Instead of a visual indicator, a user may, for example, be prompted with an audio instruction. The user may, for example, not be prompted at all after starting a program, and go through routine movements. The breathing control application may use sensors (e.g., the audio sensor 125a, the camera 125b, a lidar, radar) to analyze the user. The breathing control application may, for example, then communicate the results to the user. The breathing control application may, for example, log the improvements in a data sheet. The breathing control application may, for example, include sensory management.

FIG. 6A and FIG. 6B are block diagrams depicting an exemplary multi-dimensional immersive neuro-harmonizing system (MDINHS 600). As shown in FIG. 6A, a user 605 is using the mobile device 110 to engage in a multi-dimensional activity with the NHA 115. As shown, the SAE 135 may observe a movement of the user 605. For example, the SAE 135 may, for example, analyze the movement based on a target motion profile (TMP 610) and an actual observed motion profile (AOMP). The TMP 610 may, for example, include an index of motions, auto-corrective motion instructions, and/or motion correction prompts. Based on the TMP 610 and the AOMP, the OGE 165 and/or the GGE 160 may, for example, generate instruction prompts 615 (included in the output package 180) to the user interface 170. For example, the instruction prompts 615 may include visuals, sound, and/or movement (e.g., vibration, touch). For example, the instruction prompts 615 may include movement instructions to the user 605. The user 605 may, for example, be prompted to react to an external stimulus, such as light, vibration, and/or sound.

In some implementations, the AOMP may be observed by motion sensors. For example, the motion sensors may include cameras. For example, the motion sensors may include LiDAR. The motion sensors may, for example, include radar.

In this example, the media library 175 includes a motion media 620. The motion media 620 may, for example, interact with a neurological condition. The motion media 620 may, for example, include an index of motions, dances, martial arts, yoga instructions, and/or a cloud-based motion catalog. The motion media 620 may, for example, interact with the Internet of things (IoT). The motion media 620 may, for example, create an exercise routine, which a user may, for example, go along with the prompting and feedback from the NHA 115.

As an illustrative example, the user 605 may, for example, react to an external stimulus generated by the OGE 165. The stimulus may, for example, prompt the user to make particular target movements. The target movements may, for example, boost blood circulation, increase brain hormones such as serotonin, dopamine, endorphins, and/or oxytocin, prompt certain muscular-skeletal extensions, and prompt particular stretches. The stretches may, for example, be beneficial to treat a particular injury sustained to a particular muscle. The stretches may, for example, be used to prompt the user to perform movements corresponding to a martial art, such as Tai Chi. The stretches may, for example, prompt the user to perform a particular dance. The stretches may, for example, prompt the user to perform to go through a yoga routine.

In this example, the NHA 115 includes media package generation rules (MPGR 630). For example, the TSCE 140 may generate the target criterion 155 based on the MPGR 630. For example, the MPGR 630 may include a combination of media types associated with different predicted states and/or goals.

As shown in FIG. 6B, an exemplary MPGR 630 is shown. In the depicted example, the MPGR 630 may include a dynamic framework for generating and/or delivering various packages of media. For example, the MPGR 630 may include multiple phases of media delivery. The phases may, for example, correspond to target physiological and/or mental conditions to be induced if and/or when the media is delivered to a user.

In some implementations, for example, the MPGR 630 may include rules related to packaging and/or delivery of media. For example, the rules may include associations between media types and delivery rules. For example, the rules may be associated with specific media. The rules may, for example, be associated with specific media attributes. The rules may, for example, be associated with specific user attributes. The rules may, for example, be associated with specific delivery conditions (e.g., physiological attributes, mental attributes, realtime attributes, predicted attributes, historical attributes, environmental attributes).

By way of example and not limitation, the MPGR 630 may be embodied in a deployment environment (e.g., 855 of FIG. 8) and/or a middleware engine (e.g., 700 of FIG. 8), as disclosed at least with reference to international patent application serial no. PCT/US21/71585, titled “IMMERSIVE MEDICINE TRANSLATIONAL ENGINE FOR DEVELOPMENT AND REPURPOSING OF NON-VERIFIED AND VALIDATED CODE,” and filed by Ryan Douglas, et al. on Sep. 24, 2021, the entire contents of which are incorporated herein by reference. For example, the MPGR 630 may include rules for packaging and/or deployment of digital assets (e.g., therapeutic digital assets (TDAs), non-medical digital assets (NMDAs), such as disclosed with respect to FIGS. 1-16 of the '585 patent application. For example, the MPGR 630 may include rules for deploying digital assets in a digital deployment environment (DADE), such as disclosed in the '585 patent application. The MPGR 630 may, for example, be embodied as and/or include therapeutic modality profiles (TMPs), such as disclosed in the '585 patent application.

In the depicted example, a first target state 635 corresponds to entrainment. The media package may include, for example, a media output package (e.g., game conditions, challenge conditions, music conditions) to be generated corresponding to inducing an entrainment state for a user. The entrainment state may, for example, correspond to dopamine generation. For example, the media output package may be generated to include media corresponding to dopamine generation (e.g., selected specifically for the user, selected based on the user's historic response, selected based on other user's responses, selected based on predicted response). The dopamine generation may, for example, enable a user to physiologically be attached to continuing to engage the media.

For example, the music output package may include motion guidance (e.g., game actions, vocalization guidance).

The MPGR 630 framework may, for example, include rules for a visual-spatial interference package (VSPI). For example, in a second target state 640, the user 605 may be induced to have physical and/or emotional visual-spatial interference. For example, the OGE 165 may generate a stimuli condition to purposefully (within a prediction from the SAE 135 and TSCE 140) overwhelm meta-cognition of the user 605. For example, a goal of the second target state 640 may be selected to induce the user 605 to keep meta cognition (e.g., self-reflection) at bay to focus on the ‘hear and now’ of the situation. The MPGR 630 may cause a VSPI package to be generated based on performance, for example, of the user.

In some implementations, by way of example and not limitation, the MPGR 630 framework may be implemented in a dynamic inducement package generation system (DIPGS), such as disclosed in international patent application serial no. PCT/US2023/063720, titled “TREATMENT CONTENT DELIVERY AND PROGRESS TRACKING SYSTEM,” and filed by Ryan Douglas, et al., on Mar. 3, 2023, the entire contents of which are incorporated herein by reference. For example, the MPGR 630 may include rules for generation of a therapeutic immersive medical package (TIMP) from wild media assets (e.g., unregulated and/or non-medicinal games). For example, the MPGR 630 may be implemented in a medical media package platform (MMPG). Illustrative examples may, for example, be implemented such as disclosed at least with reference to FIGS. 1-10 of the '720 patent application. For example, the MPGR 630 may be applied to generate interventive monitoring content (IMC).

As an illustrative example, a VSPI package may present media involving mental, emotional, and/or physical challenges to the user corresponding to mental focus on the challenge at hand. For example, a tetris game in a certain difficulty range (e.g., above a certain difficulty level and/or below a certain difficulty level) may require a user to focus (e.g., almost exclusively) on the situation at hand without being frustrated and/or overly stressed. For example, the VSPI package may be generated based on current and/or historic user parameters corresponding to the media presented (e.g., response to the media; performance in a game; physiological parameters such as breath, heart rate, eye motion, pupillary response, and/or posture; textual response; vocalizations). For example, the VSPI may include media that has historically and/or is predicted to (e.g., based on other user historical responses) generate a target response (e.g., focused mentation) in the user.

In a third target state 645, the MGPR 630 framework may include rules to generate a media package such that an OGE 165 may be controlled to induce a simulated stressful condition(s). For example, a media package may be generated that induces a stress response in general and/or for the particular user. For example, an exercise game may present exercise challenges frustrating to the user. A first-person shooter game may present virtual threats to the user. A music system may, for example, present historically upsetting tunes to the user (e.g., associated with a negative response in the user such as anger, depression, despair, fear). The media package may be selected based on the user's historic response and/or predicted response (e.g., based on other user's responses and/or a profile of attributes of the user such as, for example, condition, historic experiences, diagnosis, medical state, mental state, demographics).

As an illustrative example, as shown by an illustrative breath profile plot 660 (BPM per time), the simulated stress condition may induce a physiological and/or emotional response. For example, the user may naturally begin breathing erratically, as shown by the portion of the plot 660 corresponding to the third target state 645. For example, the breathing may be elevated (e.g., above a resting rate 665). In some situations, by way of example and not limitation, the breathing may be paused (e.g., the user may hold their breath). Erratic breathing may, for example, induce a weakened and/or undesirable physical and/or mental state (e.g., helplessness, weakness, fear, anxiety).

In a fourth target state 650, the MGPR 630 framework may include rules to generate a media package configured to deliver target response training to the user. For example, the media package may induce a specific response associated with the simulated stress condition targeted in the third target state 645. For example, the training media package may be selected to induce a specific response in the user corresponding to a proper response to the simulated stress condition. For example, a training media package for the music system illustrative example may, for example, include harmonizing interventional sounds and/or visual indicia inducing the user to sing along with the stressful tune and/or to sing such that the stressful tune is converted into a calming one.

As an illustrative example, (such as, for example, corresponding to the first-person shooter response may) the MPGR 630 framework for the fourth target state 650 may include generating a media package (e.g., visual, audio, feedback) configured to induce the user to enter a controlled breathing pattern. For example, the breathing pattern may be at a rate greater than a resting rate. For example, the breathing pattern may have a BPM>5. The breathing pattern may, for example, have >6 BPM. In some examples, the breathing pattern may, for example, have up to 15 BPM. As an illustrative example, the breathing pattern may, for example, have >15 BPM. The media may, for example, be selected based on an association between the media and the user's response. For example, the MGPR 630 may include training rules for responses in a game environment (e.g., points, achievements, rewards, effects in the game) corresponding to the user following the guidance. In some examples, the rules and/or media may be selected based on the user's historic responses. The media may, for example, be selected according to the user's real-time response (e.g., to the stress condition).

In some examples, the media may, for example, be selected such that the user stabilizes erratic breathing according to a controlled pattern. For example, the user's breathing may stabilize as shown in the plot 660 corresponding to the fourth target state 650. The stabilized breath may, for example, be above the resting rate 665. For example, the stabilized breathing may correspond to a target physical and/or mental state (e.g., muscular strength, calmness, quickened perception).

In a fifth target state 655, the NHA 115 may be targeted to induce a calm state. For example, at the fourth target state 655, the user 605 may be induced to approach resting BPM. The resting BPM may, for example, include a slow BPM (e.g., 4-5 BPM). The resting BPM may, for example, be associated with a historic resting BPM of the user.

For example, the TSCE 140 may generate the target criterion 155 to generate audio based breathing guidance to help the user 605 to achieve a target breathing frequency (e.g., in the fourth target state 650 and/or the fifth target state 655). For example, the target breathing frequency in the fifth target state 655 may be selected to induce calm in the user 605 to a normal breathing frequency (e.g., 12-18 BPM). For example, the audio based breathing guidance may include humming along with a vocal track.

The media package generated for the fifth target state 655 may, for example, be selected to correspond to inducing a reflective state in the user. For example, the media may be selected to cause a user to generate serotonin. The serotonin generation after dopamine generation, the stressful condition, and the response training may, for example, induce extended recall of the response in the user and/or induce a satisfaction in successfully achieving a positive response to the stress situation. The media may, for example, be generated based on the user's historic response and/or a predicted response (e.g., other user's historic responses).

In some implementations, by way of example and not limitation, the target states (e.g., at least as related to dopamine and serotonin generation) may be implemented at least as disclosed (e.g., at least with respect to FIG. 2) in international patent application serial no. PCT/US21/71585, titled “IMMERSIVE MEDICINE TRANSLATIONAL ENGINE FOR DEVELOPMENT AND REPURPOSING OF NON-VERIFIED AND VALIDATED CODE,” and filed by Ryan Douglas, et al. on Sep. 24, 2021, the entire contents of which are incorporated herein by reference.

FIG. 7 is a block diagram of an exemplary game-induced neuro-harmonizing system (GINHS 700). In this example, the GINHS 700 includes a gaming engine 705. For example, the gaming engine 705 may, for example, include mini games that tie in with one or more predetermined/matching main games. For example, the main games may include insertion points for the gaming engine 705 to interrupt and run the mini games. For example, at an interruption point of a main game, the gaming engine 705 may, for example, insert 1-5 minute breaks of mini-games in the middle of the main game.

In some implementations, the interruption points may be static or dynamically created. Various embodiments of transitioning between the main games 710 and a mini-game generated by the game engine 705 are described with reference to PCT application serial no. PCT/US2023/063720, which shares at least one inventor with this application, titled “Treatment Content Delivery and Progress Tracking System,” filed on Mar. 3, 2023, specifically, in FIGS. 1A and 3, and [0036-42] and [0070-79]. This application incorporates the entire contents of the foregoing application(s) herein by reference.

In some implementations, the SAE 135 may observe a behavior of the user 105 in the main games 710. For example, the SAE 135 may, for example, notice that the user 105 is not implementing a particular technique (e.g., breathing) correctly. For example, the activation engine 185 may activate the gaming engine 705 and generate remedial exercises using the output package 180.

In some implementations, the main games 710 and/or the mini games may, for example, include a lowered sensory input for anxiety reduction. The gaming engine 705 may, for example, reward speaking positively instead of voicing frustration. In some implementations, the gaming engine 705 may interact with the user 105 based on a current state generated by the SAE 135. For example, based on whether a current state (e.g., engaged, unengaged, frustration, happiness, active, passive) of the user 105, the gaming engine 705 may advantageously use the OGE 165 and the target criterion 155 to generate interaction that may induce a target state of the user 105.

As an illustrative example without limitation, the gaming engine 705 may include a mini game of first person shooting genre. For example, the gaming engine 705 may configure a game control that holding breath may control a character in the game to attack. As a result, for example, the gaming engine 705 may effectively induce a non-therapeutic training for the user 105 in breathing pattern by controlling timings of appearances of adversaries in the game.

In another example, the gaming engine 705 may interrupt the main games 710 to present a challenge stage. For example, the challenge state may be generated by the OGE 165 to induce a panic state and/or stress. For example, the gaming engine 705 may generate a reward state when the user 105 responds with a calming breathing pattern. Accordingly, for example, the NHA 115 may use mini games to advantageously train a response of the user 105 in a panic state. For example, a soldier may be trained by a shooting scenario (e.g., under virtual reality equipment) to induce a calming response in a panic state. For example, the NHA 115 may advantageously replace brutal and/or unsafe training using real ammunition.

In this example, the NHA 115 also includes a community moderation engine (CME 715). The CME 715 may, for example, include third party resources to moderate user states. For example, the CME 715 may include a primary mechanism to deal with negative psychological issues (e.g., depression, frustration, anger). The CME 715 may, for example, include a help hotline, reading materials, an online library, notes left from other users, or a combination thereof. The CME 715 may, for example, include a panic emergency button for users to cut off exposure and/or limit exposure to all or some aspects of a game.

In some implementations, the CME 715 may include a musical experience (e.g., the music experience as described with reference to FIG. 1A). The musical experience may, for example, include saying words that change tones, change imagery, and/or call for interaction. The CME 715 may, in some implementations, encourage users to tap and make rhythmic sounds and shake devices. For example, the CME 715 may use the OGE 165 to generate a prompt to the user 105.

In some implementations, the CME 715 may be operably connected to other CME in a community. For example, over time the CME 715 may, for example, generate harmonized music by combining music experience (e.g., the voice clip 130) from a group of users to generate a choir music output.

In some implementations, the CME 715 may include recorded invitations to play (music, drama, plays, sports) together and collaborate. The CME 715 may, for example, include directional sound to pull players toward other players. The community moderation engine may, for example, advantageously encourage community involvement within special communities. For example, a person in a wheelchair may, for example, be encouraged to spin his chair or wheels to give his group an in-game bonus or feature.

Although various embodiments have been described with reference to the figures, other embodiments are possible.

In some implementations, the NHS 100 may include an immersive audio/visual experience utilizing player audio inputs through a breath mechanism that controls and modifies visual outputs and audio outputs (harmonizing) as a reward mechanism. For example, the NHS 100 may give players the feeling of being “part of the music” and empower them to change and enhance the experience through their input. For example, the game may be configured to give a player a base experience. The player may, for example, make their experience as visually exciting as possible through their inputs/interaction. In various embodiments, a Music Experience System (MES) may be configured to create a seamless environment where the user is entrained to the experience to such a degree that they feel as though the music is breathing with them.

In some implementations, the NHD 200 may incorporate a harmonizing loop as a way to utilize the headset's noise canceling cut-off limitation to benefit our product/experience. Various embodiments may be gamified (e.g., slightly), for example, such as with a simple mechanism to link the user's breath to the rhythm of the music.

In some implementations, the GINHS 700 may be built for specific gaming systems. In some implementations, the GINHS 700 may be broadly incorporated with popular players (e.g., such as iTunes, available from Apple Inc., Cupertino, CA; Sonos, available from Sonos, Inc., Santa Barbara, CA).

In some implementations, a player's breathing pattern may be used as a tool for co-creation of a visual. For example, a player may be provided an immersive experience just to be in there as it is. For example, a more in sync of the player's breathing may generate a more engaging visual based on the state prediction model 150.

In an illustrative example, the NHA 115 may generate a experience journey through a prerecorded song. For example, the song may be presented in a (virtual reality) 3D space. For example, the OGE 165 may generate a display at the user interface 170 of moving forwards continuously, with the pace of movement comfortable, but matching the tempo of the prerecorded song.

In various embodiments, the mobile device 110 may be coupled (e.g., directly, indirectly) to accelerometers. For example, the accelerometer may be instructed (e.g., by the guidance generated by the GGE 160) to be placed on a chest or other area of a body to detect breath. Various embodiments may, for example, advantageously solve a problem of a microphone or similar apparatus for detecting sound not being capable of picking up breath/toning sounds and/or as an additional confirmation of breathing patterns.

In some implementations, special label catalog releases may be published (e.g., the Interscope edition, the 4AD edition). As an illustrative example, some playlists in the media library 175 (e.g., audio recordings having predetermined associations with mental and/or physiological attributes to dynamically generated visualizations and/or other experiences) may be distributed as an app (e.g., on the Oculus Quest store, available through Reality Labs, Menlo Park, CA). For example, game play may be configured to make sound that affects breath. In some implementations, for example, the MES may be configured such that a player's breath/tone may affect/unlock a visual experience.

As an illustrative example, visuals may be generated (e.g., dynamically) to breathe (e.g., responsive to player input) with a player and/or instruct a player to breathe. A therapeutic element may, for example, include awareness of breath and/or controlled/rhythmic breath.

Although an exemplary system has been described with reference to FIGS. 1A-B, other implementations may be deployed in other industrial, scientific, medical, commercial, and/or residential applications.

By way of example and not limitation, a neuro-harmonizing application may be embodied in a deployment environment (e.g., 855 of FIG. 8) and/or a middleware engine (e.g., 700 of FIG. 8), as disclosed at least with reference to international patent application serial no. PCT/US21/71585, titled “IMMERSIVE MEDICINE TRANSLATIONAL ENGINE FOR DEVELOPMENT AND REPURPOSING OF NON-VERIFIED AND VALIDATED CODE,” and filed by Ryan Douglas, et al. on Sep. 24, 2021, the entire contents of which are incorporated herein by reference. For example, the media library 175 may include digital assets (e.g., therapeutic digital assets (TDAs), non-medical digital assets (NMDAs), such as disclosed with respect to FIGS. 1-16 of the '585 patent application. For example, the neuro-harmonizing application may be at least partially implemented to deploy digital assets in a digital deployment environment (DADE), such as disclosed in the '585 patent application. For example, the target state computation engine may, for example, operate at least partially as a function of therapeutic modality profiles (TMPs), such as disclosed in the '585 patent application.

In some implementations, by way of example and not limitation, a neuro-harmonizing application may be connected to and/or implemented as a part of a dynamic inducement package generation system (DIPGS), such as disclosed in international patent application serial no. PCT/US2023/063720, titled “TREATMENT CONTENT DELIVERY AND PROGRESS TRACKING SYSTEM,” and filed by Ryan Douglas, et al., on Mar. 3, 2023, the entire contents of which are incorporated herein by reference. For example, the neuro-harmonizing application 115 may be configured to generate (e.g., by the state analysis engine 135, by the target state computation engine 140, by the guidance generation engine 160, and/or by the output generation engine 165) of a therapeutic immersive medical package (TIMP) from wild media assets (e.g., unregulated and/or non-medicinal games). As an illustrative example, the application 115 may be at least partially implemented as a medical media package platform (MMPG). Illustrative examples may, for example, be implemented such as disclosed at least with reference to FIGS. 1-10 of the '720 patent application. For example, the output package 180 may be generated as an interventive monitoring content (IMC).

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Temporary auxiliary energy inputs may be received, for example, from chargeable or single use batteries, which may enable use in portable or remote applications. Some embodiments may operate with other DC voltage sources, such as 9V (nominal) batteries, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

Although particular features of an architecture have been described, other features may be incorporated to improve performance. For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device. The display device may, for example, include an LED (light-emitting diode) display. In some implementations, a display device may, for example, include a CRT (cathode ray tube). In some implementations, a display device may include, for example, an LCD (liquid crystal display). A display device (e.g., monitor) may, for example, be used for displaying information to the user. Some implementations may, for example, include a keyboard and/or pointing device (e.g., mouse, trackpad, trackball, joystick), such as by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

In an illustrative aspect, a system may include a data store that may include a program of instructions. The system may include, for example, a processor operably coupled to the data store. For example, when the processor executes the program of instructions, the processor may cause operations to be performed to automatically generate a neuro-harmonizing audible feedback package to induce a non-therapeutic state. For example, the operations may include receive an input signal from a user device. For example, the signal may include a voice clip. For example, the operations may include retrieve, from a first data store, a state prediction model configured to generate a current state as a function of audio input. For example, the operations may include identify the current state of the user device by applying the state prediction model to the input signal. For example, the operations may include retrieve, from a second data store, a state control model configured to generate a predicted state as a function of the current state and a control input. For example, the predicted state may include a target state of the user and a target probability of achieving the target state. For example, the operations may include determine a set of target criterion based on the current state. For example, the set of target criterion may include transformation parameters of a sound clip. For example, the transformation parameters may include frequency transformation, amplitude transformation, background generative transformation may include transforming a background noise of a sound clip to include sound effects, and pattern transformation. For example, the set of target criterion may be determined by applying the state control model to the current state and the target state. For example, the operations may include generate an interventional sound and an instruction as a function of the input signal and the set of target criterion. For example, the instruction may include a guidance for performing a voluntary action. For example, the target probability may be above a predetermined probability threshold. For example, the operations may include generate an audible feedback package to the user device. For example, the audible feedback package may include the interventional sound and the instruction.

The system of any of [0139-44], for example, that the voluntary action may include a vocal action.

The system of any of [0139-44], for example, that the predicted state may include a target breathing pattern, wherein the instruction may include an instruction to perform a voluntary action related to a controlled vocal track.

The system of any of [0139-44], for example, that the voluntary action may include humming along with the controlled vocal track. For example, the target breathing pattern may be induced at the target probability.

The system of any of [0139-44], for example, that the sound effects may include a background noise of a choir.

The system of any of [0139-44], for example, that the target state may be dynamically determined based on a target breath per minute.

For example, the system of any of [0139-44 may include the computer-implemented method of any of [0146-52]. For example, the system of any of [0139-44] may include the computer program product of any of [0154-160].

In an illustrative aspect, a computer-implemented method performed by at least one processor to automatically generate a neuro-harmonizing audible feedback package to induce a non-therapeutic state may include receive an input signal from a user device. For example, the method may include retrieve, from a first data store, a state prediction model configured to generate a current state associated with the input signal. For example, the method may include identify the current state of the user device by applying the state prediction model to the input signal. For example, the method may include retrieve, from a second data store, a state control model configured to generate a predicted state as a function of the current state and a control input. For example, the predicted state may include a target state of the user and a target probability of achieving the target state as a function of a set of target criterion. For example, the method may include determine a set of target criterion based on the current state. For example, the set of target criterion may include transformation parameters of a sound clip. For example, the transformation parameters may include frequency transformation, amplitude transformation, background generative transformation may include transforming a background noise of a sound clip to include sound effects, and pattern transformation. For example, the set of target criterion may be determined by applying the state control model to the current state and the target state. For example, the method may include generate an instruction to the user device as a function of the set of target criterion. For example, the instruction may include a guidance for performing a voluntary action.

The computer-implemented method of any of [0146-52], for example, that the signal may include a voice clip.

The computer-implemented method of any of [0146-52], for example, may include generate an interventional sound as a function of the voice clip and the set of target criterion. For example, the target probability may be above a predetermined probability threshold.

The computer-implemented method of any of [0146-52], for example, that the voluntary action may include a vocal action.

The computer-implemented method of any of [0146-52], for example, that the predicted state may include a target breathing pattern. For example, the instruction may include an instruction to perform a voluntary action related to a controlled vocal track.

The computer-implemented method of any of [0146-52], for example, that the voluntary action may include humming along with the controlled vocal track. For example, the target breathing pattern may be induced at the target probability.

The computer-implemented method of any of [0146-52], for example, that the sound effects may include a background noise of a choir.

For example, the computer-implemented method of any of [0146-52] may be embodied in the computer program product of any of [0154-160]. For example, the computer-implemented method of any of [0146-52] may be embodied in the system of any of [0139-44].

In an illustrative aspect, a computer program product (CPP) may include a program of instructions tangibly embodied on a non-transitory computer readable medium. For example, when the instructions may be executed on a processor, the processor may cause interactive user-specific data package generation operations to be performed to automatically generate a neuro-harmonizing audible feedback package to induce a non-therapeutic state. For example, the operations may include receive an input signal from a user device. For example, the operations may include retrieve, from a first data store, a state prediction model configured to generate a current state associated with the input signal. For example, the operations may include identify the current state of the user device by applying the state prediction model to the input signal. For example, the operations may include retrieve, from a second data store, a state control model configured to generate a predicted state as a function of the current state and a control input. For example, the predicted state may include a target state of the user and a target probability of achieving the target state as a function of a set of target criterion. For example, the operations may include determine a set of target criterion based on the current state. For example, the set of target criterion may be determined by applying the state control model to the current state and the target state. For example, the operations may include generate an instruction to the user device as a function of the set of target criterion. For example, the instruction may include a guidance for performing a voluntary action.

The computer program product of any of [0154-160], for example, that the set of target criterion may include transformation parameters of a sound clip. For example, the transformation parameters may include frequency transformation, amplitude transformation, background generative transformation including transforming a background noise of a sound clip to include sound effects, and pattern transformation.

The computer program product of any of [0154-160], for example, that the input signal may include a voice clip.

The computer program product of any of [0154-160], for example, may include generate an interventional sound as a function of the voice clip and the set of target criterion. For example, the target probability may be above a predetermined probability threshold.

The computer program product of any of [0154-160], for example, that the voluntary action may include a vocal action.

The computer program product of any of [0154-160], for example, that the predicted state may include a target breathing pattern. For example, the instruction may include an instruction to perform a voluntary action related to a controlled vocal track.

The computer program product of any of [0154-160], for example, the voluntary action may include humming along with the controlled vocal track. For example, the target breathing pattern may be induced at the target probability.

For example, the computer program product of any of [0154-160] may be embodied in the system of any of [0139-44]. For example, the computer program product of any of [0154-160] may include the method of any of [0146-52].

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A system comprising:
a data store (220) comprising a program of instructions; and,
a processor (205) operably coupled to the data store such that, when the processor executes the program of instructions, the processor causes operations to be performed to automatically generate a neuro-harmonizing audible feedback package to induce a non-therapeutic state, the operations comprising:
receive an input signal from a user device (110), wherein the signal comprises a voice clip (130);
retrieve, from a first data store, a state prediction model (150) configured to generate a current state as a function of audio input;
identify the current state of the user device by applying the state prediction model to the input signal;
retrieve, from a second data store, a state control model (140) configured to generate a predicted state as a function of the current state and a control input, wherein the predicted state comprises a target state of the user and a target probability of achieving the target state;
determine a set of target criterion (155) based on the current state, wherein the set of target criterion comprises transformation parameters of a sound clip, wherein the transformation parameters comprise frequency transformation, amplitude transformation, background generative transformation comprising transforming a background noise of a sound clip to include sound effects, and pattern transformation, and wherein the set of target criterion is determined by applying the state control model to the current state and the target state;
generate an interventional sound and an instruction as a function of the input signal and the set of target criterion, wherein the instruction comprises a guidance for performing a voluntary action, such that the target probability is above a predetermined probability threshold; and,
generate an audible feedback package (180) to the user device, wherein the audible feedback package comprises the interventional sound and the instruction.

2. The system of claim 1, wherein the voluntary action comprises a vocal action.

3. The system of claim 1, wherein the predicted state comprises a target breathing pattern, wherein the instruction comprises an instruction to perform a voluntary action related to a controlled vocal track.

4. The system of claim 3, wherein the voluntary action comprises humming along with the controlled vocal track, such that the target breathing pattern is induced at the target probability.

5. The system of claim 1, wherein the sound effects comprise a background noise of a choir.

6. The system of claim 1, wherein the target state is dynamically determined based on a target breath per minute.

7. A computer-implemented method performed by at least one processor to automatically generate a neuro-harmonizing audible feedback package to induce a non-therapeutic state, the method comprising:
receive an input signal from a user device (405);
retrieve, from a first data store, a state prediction model configured to generate a current state associated with the input signal (410);
identify the current state of the user device by applying the state prediction model to the input signal (415);
retrieve, from a second data store, a state control model (420) configured to generate a predicted state as a function of the current state and a control input, wherein the predicted state comprises a target state of the user and a target probability of achieving the target state as a function of a set of target criterion;
determine a set of target criterion based on the current state (425), wherein the set of target criterion comprises transformation parameters of a sound clip, wherein the transformation parameters comprise frequency transformation, amplitude transformation, background generative transformation comprising transforming a background noise of a sound clip to include sound effects, and pattern transformation, and wherein the set of target criterion is determined by applying the state control model to the current state and the target state; and,
generate an instruction (440) to the user device as a function of the set of target criterion, wherein the instruction comprises a guidance for performing a voluntary action.

8. The computer-implemented method of claim 7, wherein the signal comprises a voice clip.

9. The computer-implemented method of claim 7 further comprises generate an interventional sound as a function of the voice clip and the set of target criterion, such that the target probability is above a predetermined probability threshold.

10. The computer-implemented method of claim 7, wherein the voluntary action comprises a vocal action.

11. The computer-implemented method of claim 7, wherein the predicted state comprises a target breathing pattern, wherein the instruction comprises an instruction to perform a voluntary action related to a controlled vocal track.

12. The computer-implemented method of claim 11, wherein the voluntary action comprises humming along with the controlled vocal track, such that the target breathing pattern is induced at the target probability.

13. The computer-implemented method of claim 7, wherein the sound effects comprise a background noise of a choir.

14. A computer program product (CPP) comprising a program of instructions tangibly embodied on a non-transitory computer readable medium wherein, when the instructions are executed on a processor, the processor causes interactive user-specific data package generation operations to be performed to automatically generate a neuro-harmonizing audible feedback package to induce a non-therapeutic state, the operations comprising:
receive an input signal from a user device (405);
retrieve, from a first data store, a state prediction model configured to generate a current state associated with the input signal (410);
identify the current state of the user device by applying the state prediction model to the input signal (415);
retrieve, from a second data store, a state control model (420) configured to generate a predicted state as a function of the current state and a control input, wherein the predicted state comprises a target state of the user and a target probability of achieving the target state as a function of a set of target criterion;
determine a set of target criterion based on the current state (425), wherein the set of target criterion is determined by applying the state control model to the current state and the target state; and,
generate an instruction to the user device (440) as a function of the set of target criterion, wherein the instruction comprises a guidance for performing a voluntary action.

15. The computer program product of claim 14, wherein the set of target criterion comprises transformation parameters of a sound clip, wherein the transformation parameters comprise frequency transformation, amplitude transformation, background generative transformation comprising transforming a background noise of a sound clip to include sound effects, and pattern transformation.

16. The computer program product of claim 14, wherein the input signal comprises a voice clip.

17. The computer program product of claim 16, further comprises generate an interventional sound as a function of the voice clip and the set of target criterion, such that the target probability is above a predetermined probability threshold.

18. The computer program product of claim 14, wherein the voluntary action comprises a vocal action.

19. The computer program product of claim 14, wherein the predicted state comprises a target breathing pattern, wherein the instruction comprises an instruction to perform a voluntary action related to a controlled vocal track.

20. The computer program product of claim 19, wherein the voluntary action comprises humming along with the controlled vocal track, such that the target breathing pattern is induced at the target probability.
